(19) United States
(12) Patent Application Publication
(10) Pub. No.: US 2026/0157672 A1
(43) Pub. Date: Jun. 11, 2026

(54) Protected Blood Collection Glass Tube

(71) Applicant: Brigitte Paola Cardona, Deerfield, FL (US)

(72) Inventor: Brigitte Paola Cardona, Deerfield, FL (US)

(21) Appl. No.: 19/415,460

(22) Filed: Dec. 10, 2025

Related U.S. Application Data
(60) Provisional application No. 63/730,270, filed on Dec. 10, 2024.

(51) Int. Cl. A61B 5/15

(52) U.S. Cl. CPC A61B 5/150343

(57) ABSTRACT

A protected blood collection glass tube comprising an outer protective body, an inner breakable blood collection glass tube, an inner container lid which can be frictionally secured to the inner glass tube, and an outer closure cap which is configured to engage the outer protective body. The inner container lid is frictionally secured to the breakable blood collection glass tube, thereby creating a tight seal thereby providing enhanced stability and contamination prevention to the inner container. The sealed glass tube is placed into the outer protective body, the outer closure cap is introduced over the inner container lid and removably coupled to the outer protective body. The outer protective body is composed of a durable, puncture-resistant material which is adapted to retain the inner glass tube and configured to provide a vacuum seal as well as positional stabilization of the inner glass container within the outer protective body.

### CROSS-REFERENCE TO RELATED APPLICATIONS

This application claims priority from U.S. Provisional Application 63/730,270, filed Dec. 10, 2024, the disclosure of which is incorporated herein by reference.

### FIELD OF INVENTION

The present invention relates to a protected medical device, specifically a device designed to protect a glass container suitable for use for blood collection, blood separation, and the processing of blood components. More particularly, the present invention relates to a device designed for protecting breakable glass containers, such as blood collection glass tubes, which are capable of holding fluid and clotted specimens for storage, analysis, and/or processing in pharmaceutical, clinical, forensic, dental, industrial, agricultural, environmental, and veterinary applications.

### BACKGROUND OF THE INVENTION

For many years, in the biomedical science and healthcare industries, blood has been drawn to perform various tests to assess a human's health, as well as an animal's health. In certain instances, test tubes for storing, transporting, and processing blood may contain an additive or a surface coat for promoting or inhibiting clotting, depending upon the intended application. From the collected blood sample, the intended application may require the testing or processing of serum, plasma, or plasma in a fibrin clot. To accomplish this application, the blood is collected and stored in a test tube composed of glass, particularly a “plain glass blood collection tube.” In other instances, the blood is collected and stored in a plastic test tube, such as polyethylene terephthalate (PET), with the addition of a chemical additive or coating.

Traditionally, blood collection tubes were made from glass, particularly until the mid-1990s. Specifically, these blood collection tubes were composed of non-reactive glass. An advantage of these glass tubes in blood collection is that glass tubes do not require a clotting additive or silica coat because the natural surface of the glass tube inherently promoted clotting. Thus, blood contained in a glass tube would coagulate, and during centrifugation, blood separation happens and the red components of the blood fall to the bottom of the tube, leaving a plasma clot and a supernatant of serum at the upper position of the tube.

Glass test tubes and glass blood collection tubes are ideal for biomedical and health care applications requiring the retrieval of plasma clots or serum. For example, newer regenerative treatments with platelet-rich plasma (PRP) and platelet-rich fibrin (PRF), require drawing a person's blood, spinning the sample down in a centrifuge for plasma separation, retrieving plasma in PRP or clotted plasma in PRF and supernatant, and treating the person with the plasma and supernatant that may or may not be further processed. These applications are distinguished from merely drawing blood for in vitro health screenings because part of the collected sample that is platelet-rich is returned to a person's body.

More recently, modern blood collection tubes and test tubes have been made from PET-medical grade plastic which requires treatment with additives and/or coatings, especially for applications requiring centrifugation, to obtain an autologous plasma clot separation from whole blood. Indeed, plastic is almost exclusively used due to the risks posed with glass test tubes and glass collection tubes, including breakage, contamination of the sample, contamination of the laboratory, contamination and injury of the personnel handling the sample in the glass tube, and the spread of blood-borne pathogens and diseases. As a result, test and blood collection tube materials, additives, coatings, and specifications now are regulated by governing agencies such as the U.S. Food and Drug Administration (USFDA), NIOSH, CDC, and OSHA, which produced a joint advisory notice requiring the use of test and blood collections tubes that are not manufactured from glass unless such glass tubes are puncture resistant protected.

A significant advantage of using a plain glass test tube is that it does not require the use of an additive or silica coat in applications requiring the retrieval of plasma clots or serum. Conversely, plastic (PET) test tubes generally are treated with silicones and a silica coat. One serious disadvantage of such PET tubes is that inhalation and internal exposure to silica can result in fibroids, and subsequently, cancer. Further, exposure to the additives likely poses an industrial occupational hazard for those in the manufacturing sector. Individuals receiving treatments with their own serum or plasma are exposed to the additive and/or silica coat residue. In some instances, this exposure is directly into the bloodstream if the serum or plasma is injected or topically applied to an open wound or suture.

For example, the use of platelet-rich fibrin (PRF) applications in dental procedures is becoming increasing popular, mixing with bone graft material to improve bone grafting handling characteristics. The PRF procedure activates a clotting cascade to obtain a the PRF clot. This activation occurs naturally in glass tubes. When using a PET tube, a silica coat is required to activate the clotting cascade and obtain the PRF clot.

Other autologous blood plasma separation is PRP that uses PET tubes to which have been added anticoagulants to obtain plasma separation and used to mix with bone graft handling characteristics. Additional applications for PRP include hair loss, wrinkle and antiaging, and orthopedic treatments for expedited healing. These treatments typically require multiple sessions or multiple blood collection tubes. Consequently, these multiple sessions and/or multiple tubes result in greater exposure from the additives used in plastic (PET) test tubes.

Accordingly, sample exposure to additives inherently occurring with plastic (PET) tubes treated with additives or silica coats should be avoided while still conforming to government regulations for blood components' purity and safety.

### SUMMARY OF THE INVENTION

The invention of the present subject matter relates to a protected blood collection glass tube comprising an outer protective body, an inner breakable blood collection glass tube, an inner container lid which can be frictionally secured to the inner glass tube, and an outer closure cap which is configured to engage the outer protective body. In operation, the inner container lid is frictionally secured to the breakable blood collection glass tube, thereby creating a tight seal. The frictional securing provides enhanced stability and contamination prevention for protecting the inner container. Thereafter, the sealed glass tube with the inner container lid is placed into the outer protective body, the outer closure cap is introduced over the inner container lid and removably coupled to the outer protective body, for example by a threaded arrangement. The outer protective body is composed of a durable, puncture-resistant material which is adapted to retain the inner glass tube and configured to provide a vacuum seal as well as positional stabilization of the inner glass container within the outer protective body.

Accordingly, it is an object of the present subject matter to provide a cost-effective, durable, and puncture-resistant protective device for a glass blood container blood container to be used in connection with a breakable, plain glass blood container at least partially filled with a blood sample that activates clotting without the use of a silica coat.

It is another object of the present subject matter to provide a protected blood collection glass tube wherein the blood collection glass tube is capable of holding a sample which may be a bodily fluid, such as blood or serum, a soil or water sample, a pharmaceutical preparation, or a nutritional product.

It is still another object of the present subject matter to provide a protected blood collection glass tube, wherein said blood collection glass tube is configured to fit within the internal cavity of an outer protective body.

It is a yet another object of the present subject matter to provide a protected blood collection glass tube comprising a blood collection glass tube having an inner lid which prevents contamination or expulsion of a sample disposed therein, the lid being frictionally secured to the inner blood collection glass tube.

It is a further object of the present subject matter to provide a protected blood collection glass tube having an outer protective body with an outer closure cap which is configured to secure the outer protective body to the inner blood collection glass tube and the inner container lid to achieve additional frictional contact.

It is yet a further object of the present subject matter to provide a protected blood collection comprising an outer protective body, an inner blood collection glass tube, an inner container lid in the form of a rubber cap seal with a centrally disposed puncture site frictionally secured to the glass tube, and an outer closure cap, the outer closure cap configured to be introduced over said inner container lid and removably coupled to said outer protective body by a threaded arrangement in such a manner that said inner container lid extends upwardly from said outer closure cap.

It is still a further object of the present subject matter to provide a protected blood collection glass tube which can be disposed in a single sterile pack and be configured for an intended application, such as platelet-rich fibrin applications in dental and oral surgery procedures.

These and other objects of the present subject matter are accomplished by providing a protected blood collection glass tube comprising an inner blood collection tube disposed within the cavity of an outer protective body composed of polyethylene terephthalate (PET) puncture resistant material. This outer protective body may be fixable or removably attachable by frictional contact to an inner glass container, without any communication that allows fluid or air passage between the inner container and outer protective body.

### BRIEF DESCRIPTION OF DRAWINGS

FIG. 1 is an exploded view of the protected blood collection glass tube of the present subject matter.

FIG. 2 is a side perspective view of the protected blood collection glass tube illustrating the inner container with an inner container lid frictionally secured thereto the outer protective body, and the outer closure cap.

FIG. 3 is a side perspective view of the protected blood collection glass tube illustrating the inner container with an inner container lid frictionally secured thereto, introduced into a plastic container.

FIG. 4 is a side perspective view of the assembled protected blood collection glass tube of the present subject matter.

FIG. 5 is a perspective view of the protected blood collection glass tube of the present subject matter packaged in a single-use, sterile pack.

It is to be understood that the drawings are not necessarily to scale. Rather, the drawings are merely representations, not intended to portray specific parameters of the present subject matter unless specified otherwise. The drawings are intended to depict exemplary embodiments of the present subject matter, and therefor should not be considered limiting in scope. In the drawings, like numbering represents like elements. Further, certain elements in some figures may be omitted, or not illustrated to scale, for illustrative clarity. Any cross-sectional views may be in the form of “slices”, or “near-sighted” cross-sectional views, omitting certain background lines that may otherwise be visible in a “true” cross-sectional view, for illustrative clarity. In some instances, where multiple elements are present, only a single element may be labeled or multiple elements may have the same label to indicate that the label applies to all elements of the same type.

### DETAILED DESCRIPTION OF THE INVENTION

In the following description, like reference numbers are used to identify like elements. Furthermore, the drawings are intended to illustrate major features of exemplary embodiments in a diagrammatic manner. The drawings are not intended to depict every feature of every implementation nor relative dimensions of the depicted elements and are not drawn to scale. In the following description, numerous specific details are set forth to clearly describe various specific embodiments disclosed herein. One skilled in the art, however, will understand that the presently claimed invention may be practiced without all the specific details discussed below. In other instances, well known features have not been described so as not to obscure the invention.

The terms “inner container,” “inner tube”, “inner glass tube”, derivatives thereof and similar terms may be used throughout the specification and it is to be understood that such terms are interchangeable and all refer to an inner blood collection glass tube. Similarly, the terms “plastic container”, “plastic body”, “outer body”, “outer protective body”, “protective device”, derivatives thereof and similar terms may be used throughout the specification and it is to be understood that such terms are interchangeable and all refer to the protective plastic outer tube of the present subject matter.

Also, it is to be understood that the phraseology and terminology used herein is for the purpose of description and should not be regarded as limiting. The use of the terms “including”, “comprising”, “having”, and variations thereof herein is meant to encompass the items listed thereafter and equivalents thereof as well as additional items.

Reference throughout this specification to “one embodiment” or “an embodiment” means that a particular feature, structure, or characteristic described in connection with the embodiment is included in at least one embodiment. Thus, the use of the phrases “in one embodiment” or “in an embodiment” throughout the specification are not necessarily referring to the same embodiment. Further, particular features, structures, or characteristics may be combined in any suitable manner in one or more embodiments. Headings provided herein are for convenience only and do not interpret the scope or meaning of the claimed invention. The present disclosure is directed to overcoming or at least reducing the effects of one or more of the problems presented above.

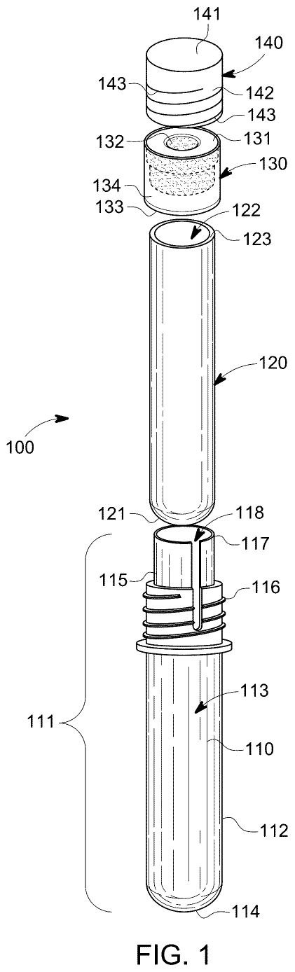

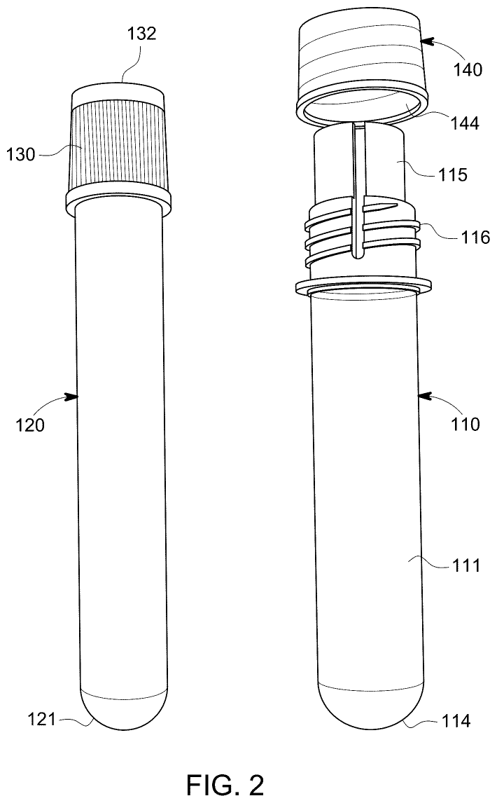

As shown in FIG. 1 and FIG. 2, the present subject matter is directed to a protected blood collection glass tube 100 comprising an outer protective body 110, a blood collection glass tube 120 disposed within the outer protective body, an inner container lid 130 configured to be frictionally secured to the blood collection tube 120, and an outer closure cap 140 which is configured to be introduced over the inner container lid 130 and removably coupled to the outer protective body 110.

Referring now to FIG. 1, the outer protective body 110 is in the form of a elongated tubular housing 111 having an outer surface 112, an internal cavity 113, a closed lower end 114, and upper open end 115 having a threaded arrangement 116 on the outer surface proximal to said upper open end, a rim 117, about the periphery of the upper open end 115, and an opening 118 disposed at the open end 115. The addition of the rim 117 allows for the validation of the integrity of the inner glass tube. The lower end 114 may be configured with any suitable shape. In the illustrated embodiment, the closed lower end 114 is rounded. The plastic outer body is dimensioned to receive the inner glass tube 120 within the internal cavity 113.

The outer protective body is composed of a shatter-resistant material which may be a molded or extruded plastic material. In one embodiment, the outer protective body is composed of a durable plastic, such as polyethylene terephthalate (PET). Other suitable materials for use in manufacturing the outer protective body include, but are not limited to, latex, ceramic, fiberglass, metal, flexi-glass, rubber, melamine, tempered glass, polypropylene, carbon, glazed stoneware, metallic glass, and combinations thereof. The material selected for making the outer protective body must be a shock-resistant material, meaning that the outer protective body can sustain an impact without damage to the inner glass tube or to the biological, medical, or chemical sample contained within the inner glass tube, or to any other liquids, food, or other consumable samples.

In one embodiment, the outer protective body is translucent or transparent to allow visibility of the sample contained within the blood collection glass tube 120 when the glass tube is disposed within the internal cavity 113. A transparent outer protective body provides quality control by enabling validation of the integrity of the glass tube, including determining any glass tube breakage. The plastic outer protective body is designed to prevent breakage of the inner glass tube disposed in the internal cavity, thereby preventing destruction and contamination of the sample contained within the glass tube. In one embodiment, the outer protective body may be removably coupled to the blood collection glass tube. In one embodiment, the outer protective body can be fixedly attached to the inner glass container for a single use. The protective body preferably is dimensioned to fit within a standard centrifuge and test tube holder.

The blood collection glass tube 120 is dimensioned to be introduced into the internal cavity 113 of the outer protective body 120. The glass tube 120 is configured to receive a biological fluid, medical fluid, food sample, or chemical sample. The glass tube is composed of a non-contaminating material, preferably a non-reactive glass material, conventionally referred to as a plain glass blood collection tube. The benefit of using a non-reactive glass material is that blood coagulation is promoted within the glass collection tube, particularly when the biological, medical, or chemical sample is a blood sample. The sample collected may comprise a solid, liquid or gas. The sample is deposited into the inner container before or after the inner container is introduced into the inner cavity of the outer protective body, if the outer protective body is removable coupled to the inner container. Further, it is contemplated to be within the scope of the present invention that the inner container may further comprise at least one additive, such as an SFS coating, a gel, a clot activator, a separator, or an anticoagulant compound. It also is contemplated to be within the scope of the present invention that other materials may be used in the manufacture of the inner container, suitable examples of which include glass, ceramic, porcelain, and plastics.

In one embodiment, the inner glass container 120 is configured to be processed in a centrifuge when assembled within the outer protective body 110. In one embodiment, the inner container is configured to be used in the manufacture of platelet rich fibrin (PRF). In one embodiment, the inner glass container 120 is configured to be used in In Vitro Diagnostic (IVD) tests.

As shown in FIG. 1 and FIG. 2, the inner glass tube 120 comprises a closed lower end 121 and an upper open end 122 having an outer rim 123. A sample is introduced into the upper open end 122. The glass tube 120 being dimensioned to fit within the internal cavity 113 of the plastic protective body. An inner container lid 130 is provided to seal the sample within the blood collection tube 120. The inner container lid 130 may be in the form of a rubber seal having a top surface 131, a puncture site 132 centrally disposed in the top surface, and a lower end 132 having a lower end rim 133, the inner container lid 130 being designed and configured such that the lower end rim 133 is introduced onto an over the outer rim 123 of the inner glass tube 120, thereby effecting a seal by frictionally securing the lid 130 on the glass tube 120. This frictional contact ensures enhance stability and contamination prevention for protecting the sample within the inner glass tube. The frictionally sealed inner glass container 120 is shown in FIG. 2.

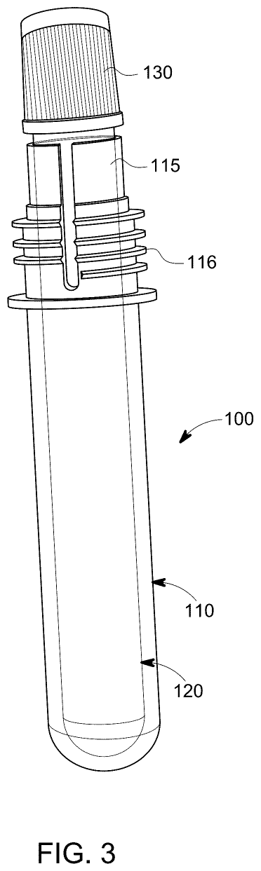

The plastic outer protective body 110 is sized to receive the sealed inner glass container 120 as shown in FIG. 3. For illustrative purpose, the outer protective body may be about 15.0 cm in length in the inner container may be from about 10.0 cm to about 12.0 cm in length. In one embodiment, the outer protective body and the inner glass tube may be of equivalent lengths. As will be obvious to those skilled in the art, conventional sizes and shapes may be implemented as necessary.

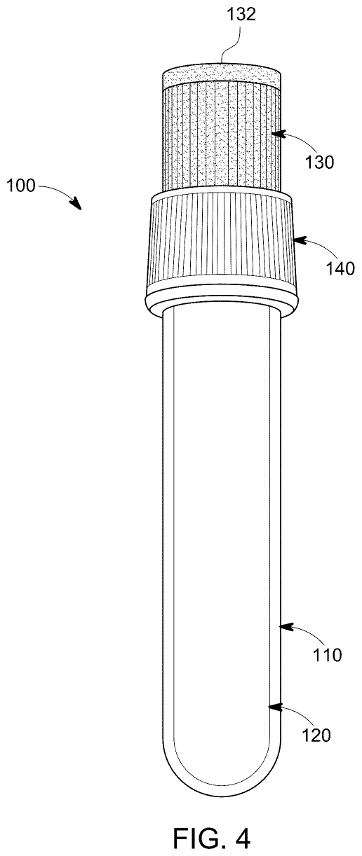

Once a sample has been collected in the blood collection glass tube 120 and sealed with the inner container cap 130, the thus assembled sealed glass tube is introduced into the internal cavity 113 of the outer protective body 110. The sealed blood collection glass tube 120 is arranged in a containment position within the outer protective body by means of an upper closure cap 140 as shown in FIG. 4. As best seen in FIG. 1 and FIG. 2, the upper closure cap is in the form of a screw nut comprising an open top end 141, a circular side wall 142 with an internal threaded arrangement 143, and a lower open end 144. The configuration of the upper closure cap 140 forms an interface with the outer protective body 110 as the internal treaded arrangement 143 corresponds to the outer threaded arrangement 116. In operation, once the sealed blood collection glass tube is within the internal cavity, the outer closure cap 140 is introduced over the inner container lid 130 and is removably coupled to the plastic outer tube by the threaded arrangement. The outer closure cap frictionally slides over the inner container lid such that the at least part of the inner container lid 130 protrudes upwardly from the sealed container as shown in FIG. 4. In this manner, the top surface 131 and puncture site 132 are accessible to the user.

Preferably, the protected blood collection glass tube should be treated like any other collection tube or test tube with safety precautions including, for example, placing the protected device in a holder when not in use, or when placing an inner container lid, or outer closure cap on the protected device.

The protected blood collection glass tube of the present subject matter is designed to ensure that any sample container within the blood collection glass tube will not escape from inner container and into the internal cavity as a first seal is formed between the inner container lid and the inner glass tube and a second seal is formed between the outer closure cap, the inner container lid, and the outer protective body.

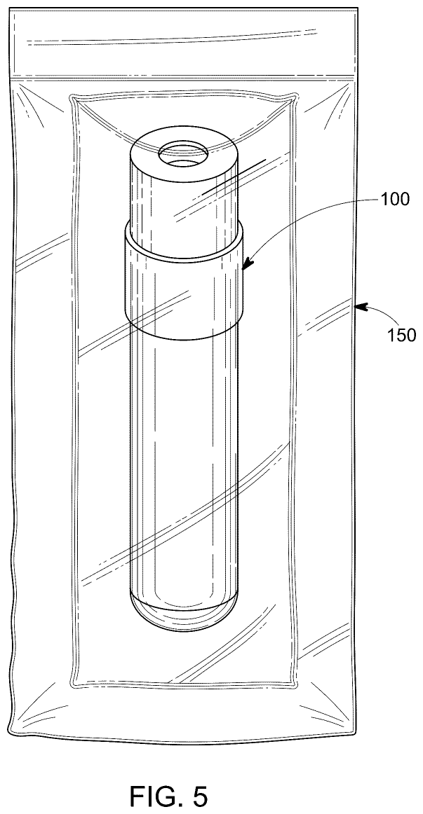

As shown in FIG. 5, the protected blood collection glass tube of the present subject matter may be packaged in a single-use, sterile pack 150. In particular, the protected blood collection glass tube can be disposed in a single sterile pack for a specific application, such as platelet-rich fibrin (PRF) applications in dental and oral surgery procedures. Specialized sterile packs may be customized according to the intended application. For example, dental and oral surgery procedures using PRF may require one to six protected devices for single use, whereas environmental applications may require a pack of twelve protected tubes for various sample sites.

While several illustrative embodiments of the invention have been shown and described, numerous variations and alternative embodiments will occur to those skilled in the art. Such variations and alternative embodiments are contemplated and can be made without departing from the scope of the invention as defined in the appended claims.

The foregoing detailed description of exemplary and preferred embodiments is presented for purposes of illustration and disclosure in accordance with the requirements of the law. It is not intended to be exhaustive nor to limit the invention to the precise form(s) described, but only to enable others skilled in the art to understand how the invention may be suited for a particular use or implementation. The possibility of modifications and variations will be apparent to practitioners skilled in the art. No limitation is intended by the description of exemplary embodiments which may have included tolerances, feature dimensions, specific operating conditions, engineering specifications, or the like, and which may vary between implementations or with changes to the state of the art, and no limitation should be implied therefrom. Applicant has made this disclosure with respect to the current state of the art, but also contemplates advancements and that adaptations in the future may take into consideration of those advancements, namely in accordance with the then current state of the art. It is intended that the scope of the invention be defined by the Claims as written and equivalents as applicable. Reference to a claim element in the singular is not intended to mean “one and only one” unless explicitly so stated. Moreover, no element, component, nor method or process step in this disclosure is intended to be dedicated to the public regardless of whether the element, under the provisions of 35 U.S.C § 112, sixth paragraph, unless the element is expressly recited using the phrase “means for . . . ” and no method or process step herein is to be construed under those provisions unless the step, or steps, are expressly recited using the phrase “step(s) for . . . .”

1. A protected blood collection glass tube comprising:
a. a protective outer body in the form of an elongated housing having a first closed lower end, a first open upper end, a first rim disposed about the periphery of said open upper end, and an internal cavity, said first open upper end having a threaded exterior section near said first open upper end;
b. an inner container having a second closed lower end, a second open upper end, and a second inner rim disposed about the periphery of said second open upper end, said inner container being dimensioned to fit within said internal cavity,
c. an inner container lid having a top surface, a puncture site centrally disposed in said top surface, a lower end with a lower end rim, said inner container lid configured to frictionally seal said second open upper end of said inner container, and
d. an outer closure cap having an open top end, a circular side wall having a threaded internal section, and an open lower cap end, wherein said inner container lid frictionally seals said inner container to form a sealed inner container having a vacuum seal, said sealed inner container is introduced within said internal cavity of said protective outer body and said outer closure cap frictionally slides over said inner container lid and is removably coupled to said protective outer body by threaded engagement, said puncture site protruding from said outer closure cap.

2. The protected blood collection glass tube in accordance with claim 1, wherein said inner container is composed of a plain glass material.

3. The protected blood collection glass tube in accordance with claim 2, wherein said inner container is a blood collection glass tube.

4. The protected blood collection glass tube in accordance with claim 3, wherein said inner container is configured to be used for the separation of blood components to obtain a platelet rich fibrin clot.

5. The protected blood collection glass tube in accordance with claim 1, wherein said outer protective body is composed of a puncture-resistant material.

6. The protected blood collection glass tube in accordance with claim 5, wherein said outer protective body is composed of a material selected from the group consisting of polyethylene terephthalate, plastic, latex, ceramic, fiberglass, metal, flexi-glass, rubber, melamine, tempered glass, polypropylene, carbon, glazed stoneware, metallic glass, and combinations thereof.

7. The protected blood collection glass tube in accordance with claim 6, wherein said outer protective body is composed of polyethylene terephthalate.

8. The protected blood collection glass tube in accordance with claim 5, wherein said outer protective body is transparent.

9. The protected blood collection glass tube in accordance with claim 8, wherein said transparent outer protective body ensures quality control by enabling validation of the integrity of the glass tube, including determining any glass tube breakage.

10. The protected blood collection glass tube in accordance with claim 1, wherein said inner container is composed of a material selected from the group consisting of glass, ceramic, and porcelain.

11. The protected blood collection glass tube in accordance with claim 1, wherein said inner container further comprises at least one additive selected from the group consisting of SFS coating, a gel, a clot activator, a separator, and an anticoagulant compound.

12. The protected blood collection glass tube in accordance with claim 1, wherein said inner container lid is in the form of a rubber seal having a top surface with a puncture site centrally disposed in said top surface of said rubber seal.

13. The protected blood collection glass tube in accordance with claim 1, which is packaged in a single use sterile pack.

14. The protected blood collection glass tube in accordance with claim 13, wherein said inner container is configured to be used for the separation of blood components to obtain a platelet rich fibrin clot.

15. The protected blood collection glass tube in accordance with claim 14, wherein said inner container is a blood collection glass tube.

16. The protected blood collection glass tube in accordance with claim 15, wherein said inner container is configured to be used for the separation of blood components to obtain a platelet rich fibrin clot (PRF) for application in dental and oral surgery procedures.
